(19) United States
(12) Patent Application Publication
(10) Pub. No.: US 2023/0143893 A1
(43) Pub. Date: May 11, 2023

(54) ISOLATION AND PURIFICATION OF EXOSOMES FOR REGENERATIVE MEDICINE

(71) Applicant: Avem Holdings LLC, Las Vegas, NV (US)

(72) Inventors: Travis H. Bird, Las Vegas, NV (US); Tammy Luttrell, Las Vegas, NV (US); Michael Howell, Las Vegas, NV (US); Samantha Rosenberry, Las Vegas, NV (US)

(21) Appl. No.: 17/913,301

(22) PCT Filed: Mar. 23, 2021
(86) PCT No.: PCT/US2021/023786

§ 371 Date: Sep. 21, 2022

Related U.S. Application Data
(60) Provisional application No. 63/012,819, filed on Apr. 20, 2020.
(60) Provisional application No. 62/993,531, filed on Mar. 23, 2020.

(51) Int. Cl. A61K 35/51

(52) U.S. Cl. CPC A61K 35/51; C12N 2310/141

(57) ABSTRACT

The present application provides a method of isolating highly purified and characterized exosomes from tissue and cellular sources for the purpose of regenerative medicine as well as a composition including an exosome or an exosomal compound or component. The composition is used for the therapeutic treatment of various physiological damages and diseases, including skin or cutaneous damages/diseases, cardiovascular diseases, ophthalmic diseases, neurological diseases, viral diseases, and cancer among other damages and diseases.

### CROSS REFERENCE

This application claims the benefit of US Provisional Patent Application No. 62/993,531, filed Mar. 23, 2021, and U.S. Provisional Patent Application No. 63/012,819, filed Apr. 20, 2020, which are incorporated by reference herein in its entirety.

### FIELD

The present provisional application provides methods for the isolation and purification of exosomes from animal tissue, in particular human tissue, and peripheral blood for use in regenerative medicine and compositions including exosomes and/or exosomal components for the therapeutic treatments of diseases and conditions. This disclosure relates to mesenchymal stem cell exosomes (MSC-EX) in the pharmaceutical composition COVIXO.

### BACKGROUND

Stem cells are unspecialized cells of the body and have the ability to self-renew and differentiate into many types of cells. The properties of stem cells have prompted their use as therapies in many therapeutic cases including but not limited to severe injuries, genetic and degenerative disorders, and hematologic malignancies. Stem cell transplantation has been shown to be an effective therapeutic approach to replace lost tissue with new cellular material or to facilitate the regeneration of damaged or diseased tissues through paracrine cell signaling. The relative success of these clinical studies has prompted further investigation and characterization of stem cells as potential therapeutics.

Stem cells can be further separated into subcategories based on tissue origin, stage of development (e.g., adult, embryonic), size, and properties. Examples of stem cells are outlined in Table 1.

TABLE 1
Stem Cell

Embryonic Stem Cell
Pluripotent Stem Cell
Induced Pluripotent Stem Cell
Mesenchymal Stem Cell
Hematopoietic Stem Cell

Basic science and clinical studies have indicated that utilization of mesenchymal stem cells (MSCs) among other stem cells is a promising strategy for cutaneous regeneration and mitigation of inflammatory responses across many tissue types. (Rahaman, et al.). MSCs have been shown to migrate to injured tissues where they act through paracrine signaling to release biologically active molecules that affect the proliferation, migration and survival of neighboring cells. (Pittenger, et al.). Further, immunomodulation by MSCs accelerates cutaneous regeneration by promoting formation of a well-vascularized granulation matrix, increasing proliferation and migration of skin cells while inhibiting apoptosis. Despite the benefits of MSCs, potential adverse events have prompted the need for further purified and characterized cell-free products. (Vulliet, et al.). For example, graft versus host disease is a potential adverse effect.

Most cells are thought to release extracellular vesicles. Therefore, extracellular vesicles are found in many different biofluids including blood, breast milk, saliva, urine, and cerebrospinal fluid. The extracellular vesicles contain proteins, metabolites (including proteins and lipids), and nucleic acids (e.g., microRNA and mRNA) that reflect the cellular origin and function of the exosome. Extracellular vesicles function in intercellular communication through the transfer of proteins and RNA affecting systemic processes such as immune function (Raposo, et al.) and inflammation, (Robbins, et al.) as well as a host of disease- and organ-specific processes. Considering their importance in intercellular communication, their role in therapeutic effect could be applied in a cell-free system, in particular with respect to exosomes or exosomal products derived from stem cells.

In fact, recent studies have demonstrated that biological activity exhibited by MSCs may be due to the release of small extracellular vesicles (sEVs) during the implantation process. These sEVs—which include exosomes—carry a cargo enriched in proteins, micro-RNAs, and metabolites/factors having immunomodulatory and other therapeutic-related activities. Exosomes derived from stem cells can deliver and transfer beneficial effects by providing proteins, bioactive lipids, metabolites and nucleic acid cargo to neighboring diseased or injured cells, thereby promoting the activation of regenerative and reparative cell-programs. As mentioned in the foregoing paragraph, exosomes play key roles in cell-cell communication, acting both proximally and systemically. Exosomes may regulate many physiologic and pathologic processes by affecting the survival, migration, proliferation and gene expression of recipient cells. The targeted gene expression of the recipient cells is believed to be reprogrammed by the exosomes cargo, specifically microRNA (miR), messenger RNA (mRNA) and proteins.

The extracellular vesicles are generally classified based on size and biogenesis. For example, exosomes are defined as having a diameter of less than 150 nm, whereas ectosomes or microparticles (microvesicles) are defined as having a diameter up to 1000 nm. Regarding the formation of extracellular vesicles, exosomes are described as being derived from multivesicular bodies (MVBs) and a wide range of cells. Thus, exosomes are generally defined to be about 20 nm to about 150 nm in size and are small membrane vesicles that originate from the internal budding of the late endosomal membrane. Exosomes have several unique characteristics including the classic type of spherical or dish morphology, lipid bilayer, density of about 1.13-1.19 g/mL and certain enriched protein markers—including tetraspanisn, TSG101, and Hsp70. (Thery et al.)

Improved purification and characterization of exosomes is needed for developing new and more effective therapeutics that are aimed at providing therapeutic effects, including modulating immune and inflammatory pathways while stimulating regenerative medicine. In addition, purified exosomes and/or exosomal products are effective for therapeutic effects. This application is directed to that need and others.

### SUMMARY

The present application provides methods of isolating and purifying exosomes from various tissue and cellular sources and the compositions including exosomes and/or exosomal components for the therapeutic treatments of diseases and conditions.

In some embodiments, exosomes may be isolated and purified from placental tissue, umbilical cord tissue, and Wharton's Jelly (umbilical cord tissue), 2D cell culture, 3D cell culture, among other sources.

In some embodiments, exosomes may be isolated and purified from peripheral blood, and cord blood, etc., among other sources.

In some embodiments, the exosomes may be purified from pluripotent stem cells, embryonic stem cells, induced pluripotent stem cells, mesenchymal stem cells, hematopoietic stem cells, and extracellular vesicles, among other sources.

In some embodiments, the isolated exosomes and/or exosomal products are in a composition for the treatment.

### BRIEF DESCRIPTION OF DRAWINGS

FIG. 1 is a schematic of extracellular vesicles including exosomes.

FIG. 2 illustrates the steps for the purification and isolation of exosomes or exosomal 5 products from MSC.

FIG. 3 exhibits the characteristic of an exemplary Sample A purified from the disclosed method of purification showing the constituent portions from the cytoplasm, cytosol, membrane, nucleus, extracellular portion, and organelle lumen. FIG. 3 also identifies the proportion of the proteins with respect to their molecular function and biological process.

FIG. 4 exhibits the characteristic of an exemplary Sample B purified from the disclosed method of purification showing the constituent portions from the cytoplasm, cytosol, membrane, nucleus, extracellular portion, and organelle lumen. FIG. 4 also identifies the proportion of the proteins with respect to their molecular function and biological process.

FIG. 5 exhibits a Ven Diagram comparing Sample A and Sample B using a Liquid Chromatography—Mass Spectrometry (LC/MS) analysis. FIG. 4 that both samples have commonalty with respect to most of their constituent proteins/molecules (1916), whereas Sample A only has 319 unique proteins/molecules and Sample B only has 215 unique proteins/molecules.

### DETAILED DESCRIPTION

The present application provides, inter alia, a method of isolating highly purified and characterized exosomes from tissue and cellular sources for the purpose of regenerative medicine as well as a composition including an exosome or an exosomal compound or component. The composition is used for the therapeutic treatment of various physiological damages and diseases, including skin or cutaneous damages/diseases, cardiovascular diseases, ophthalmic diseases, neurological diseases, viral diseases, and cancer among other damages and diseases.

The term “Embryonic stem cells” (ES cells or ESCs) as used herein refers to pluripotent stem cells derived from the inner cell mass of a blastocyst, an early-stage pre-implantation embryo Human embryos reach the blastocyst stage 4-5 days post fertilization, at which time they consist of 50-150 cells. Isolating the embryoblast, or inner cell mass (ICM) results in destruction of the blastocyst, a process which raises ethical issues, including whether or not embryos at the pre-implantation stage should have the same moral considerations as embryos in the post-implantation stage of development.

The term “Pluripotent stem cells” as used herein is a type of cells capable of unlimited, undifferentiated proliferation in vitro and still maintain the capacity to differentiate into a wide variety of somatic cells. In this capacity, pluripotent stem cells have widespread clinical potential for the treatments of heart disease, diabetes, spinal cord injury, and a variety of neurodegenerative disorders.

The term “Induced pluripotent stem cells” (also known as iPS cells or iPSCs) as used herein is a type of pluripotent stem cell that can be generated directly from a somatic cell.

The term “Hematopoietic stem cells” as used herein is a type of stem cells that give rise to other blood cells. This process is called hematopoiesis. This process occurs in the red bone marrow, in the core of most bones. In embryonic development, the red bone marrow is derived from the layer of the embryo called the mesoderm.

The term “Mesenchymal Stem Cells (MSC)” as used herein refers to multipotent stromal cells that can differentiate into a variety of cell types, including osteoblasts (bone cells), chondrocytes (cartilage cells), myocytes (muscle cells) and adipocytes (fat cells which give rise to marrow adipose tissue). (Ankrum, et al.). MSCs are adult stem cells traditionally found in the bone marrow. However, mesenchymal stem cells can also be isolated from other tissues including cord blood, peripheral blood, fallopian tube, and fetal liver and lung. Multipotent stem cells, MSCs differentiate to form adipocytes, cartilage, bone, tendons, muscle, and skin. Mesenchymal stem cells are a distinct entity to the mesenchyme, embryonic connective tissue which is derived from the mesoderm and differentiates to form hematopoietic stem cells.

MSC based therapy has achieved positive effects in various animal models of diseases and several human clinical trials. MSC's have demonstrated favorable therapeutic effects in diseases like bone fracture, traumatic brain injury, stroke and myocardial infarction. (Wu, et al.). Numerous basic science and clinical studies have indicated that MSCs are a promising strategy for cutaneous regeneration among other regenerations and mitigation of inflammatory responses across many tissue types. MSC are easily isolated and have been demonstrated to home to injured tissues undergoing subsequent differentiation to repair and replace damaged cells. MSCs are reported to accelerate cutaneous regeneration by modulating the inflammatory response, promoting formation of a well-vascularized granulation matrix, increasing proliferation and migration of skin cells while inhibiting apoptosis. However, emerging evidence has shown that transplanted MSCs are not without risk. Vulliet, et. al. reported myocardial micro-infarction after intra-arterial injection of MSCs. These results among others set the foundation for the development of cell-free treatment that retains the desirable effects of the MSCs. Currently, MSC's are believed to effect cells through paracrine signaling; such that MSCs release biologically active molecules that affect the proliferation, migration and survival of neighboring cells. (Yao, et al.).

The term “Micelle” is an aggregate (or supramolecular assembly) of surfactant molecules dispersed in a liquid colloid. A typical micelle in aqueous solution forms an aggregate with the hydrophilic “head” regions in contact with surrounding solvent, sequestering the hydrophobic single-tail regions in the micelle center.

The term “Wharton's jelly” (substantia gelatinea funiculi umbilicalis) as used herein refers to a gelatinous substance within the umbilical cord, largely made up of mucopolysaccharides (hyaluronic acid and chondroitin sulfate). It acts as a mucous connective tissue containing some fibroblasts and macrophages and is derived from extra-embryonic mesoderm.

The term “Exosomes” as used herein refers to biologically active signaling vesicles responsible for the paracrine effects of MSCs. They are also considered nano biovesicles. In the field of regenerative medicine, stem cells transplantation has been demonstrated to be an effective therapeutic approach that replaces lost tissue with new cellular material or facilitates the regeneration of damaged or diseased tissues through paracrine cell signaling. Exosomes secreted by MSCs may be the signal for the biological activity observed with implantation of the stem cells themselves.

Most bodily fluids contain exosomes. (Alenquer, et al.). Their contents have been shown to change in various diseases including viral infections, neurodegenerative diseases (prions, Alzheimer, Huntington disease), and cancer, and hence exosomes are being intensively investigated also as a source of novel biomarkers. (Lin, et al.) In recent years, a plethora of reports and reviews has explored several functions of exosomes in mediating intercellular communication, immune system functions, development and differentiation, neuronal function, cell signaling, regeneration, and several steps in viral replication. (Sharma, et al.).

Consistent with the concentration of MSC therapeutic potency in its secretion, a significant proportion of MSC immune potency resides in the small extracellular vesicles (sEVs) secreted by MSCs. These sEVs, which also include exosomes, are designed to carry a large cargo enriched in proteins, micro-RNA, have immunomodulatory activities. Exosomes derived from stem cells can deliver and transfer some beneficial outputs of stem cells including proteins, bioactive lipid and nucleic acid cargo to neighboring diseased or injured cells, thereby promoting the activation of regenerative and reparative cell-programs. This supports the paracrine hypothesis that the benefits conveyed from MSCs as a component of regenerative medicine are largely based on exosomes secreted by the stem cells rather than the MSCs themselves. Exosomes are the mechanism, representative “word-packets” delivered to neighboring cells in the “language-of-the-cells”.

Exosomes are now known to play key roles in cell-cell communication, acting proximally and systemically, paracrine signaling Exosomes may regulate may physiologic and pathologic processes by affecting the survival, migration, proliferation and gene expression of recipient cells. The targeted gene expression of the recipient cells is believed to be reprogrammed by the exosomes cargo, specifically microRNA (miR), messenger RNA (mRNA) and proteins. (Zhang, et al.).

Exosomes are generally defined to be 20 nm to 150 nm in size and are small membrane vesicles that originate from the inside budding of the late endosomal membrane. Exosomes have several unique characteristics including the classic type of spherical or dish morphology, lipid bilayer, density of 1.13-1.19 g/mL and certain enriched protein markers including tetraspanisn, TSG101, and Hsp70. (Thery, et al.).

Exosomes can also be used as biomarkers and possible diagnosis, both for prognostics and as predictors of disease trajectory. Exosomes are found widely in the body fluids including blood, urine, saliva and breast milk.

The term “Exosomal Composition or Exosomal Product” as used herein refers to the molecules or the cargo within the exosome. It includes surface molecules of the exosome. The term refers to all molecules that are part of the exosomes or form the exosomes. They include all molecules attached directly or indirectly to exosomes.

The term “Exosome Isolation” as used herein refers to methods for isolation of exosomes from both body fluids and MSCs. Primarily, exosome isolation includes high-performance liquid chromatography (HPLC), ultracentrifugation and immunebead isolation. The issue for recovery of exosomes is one of preservation of the complex cargo.

The term “Exosome Cargo Characterization’ as used herein refers to the multiple means to validate the specific exosome composition, purity and concentration including the following which are conducted through a 3^{rd} party laboratory: (1) identification of exosomes by 6 different CD cell-surface markers which are indicative of the MSC from which the exosomes originate; (2) processing that yields 50 to 100 nm vesicles, verified by Nanocyt technology; (3) identification of miRNA contained in the exosomes; (4) protein identification via ELISA (Enzyme-Linked Immunosorbent Assay); and/or (5) exosome count validation/microliter.

The cargo carried by exosomes includes the following: mRNA, proteins and MicroRNAs (miRNAs. short (20-24 nt) non-coding RNAs that are involved in post-transcriptional regulation of gene expression in multicellular organisms by affecting both the stability and translation of mRNAs; miRNAs are transcribed by RNA polymerase II as part of capped and polyadenylated primary transcripts (pri-miRNAs) that can be either protein-coding or non-coding.

The term “Scaffolding” as used herein refers to materials that have been engineered to cause desirable cellular interactions to contribute to the formation of new functional tissues for medical purposes. Cells are often ‘seeded’ into these structures capable of supporting three-dimensional tissue formation. Scaffolds mimic the extracellular matrix of the native tissue, recapitulating the in vivo milieu and allowing cells to influence their own microenvironments. They usually serve at least one of the following purposes: allow cell attachment and migration, deliver and retain cells and biochemical factors, enable diffusion of vital cell nutrients and expressed products, exert certain mechanical and biological influences to modify the behavior of the cell phase.

Scaffolding serves two main purposes: Differentiation of 3D MSC's into specific lines utilizing 2D Cultures/scaffolds and facilitation of regeneration and healing in various integumentary and bone tissues. Combination of exosome products with biologically active scaffold materials for wound care/skin regeneration, bone regeneration. Biologically active materials include nanofibrous polymers, hyaluronic acid, chitosan, hydrogel, laminin, collagen, alginate among other materials. For example, the addition of nitric oxide (NO)+chitosan increases the release of exosomes from placental MSC. These MSCs are pro-angiogenic and pro-migratory resulting in increased vasoendothelial growth factor (VEGF) and increased miR126 with ischemic muscle tissue protected.

Diseases to be Treated Using Exosomes

The description and list of the diseases and damages is not exhaustive.

Cutaneous Damages/Diseases

The skin's function is often compromised as a result of damage from trauma, burns, chronic or diabetic wounds. The destruction of the integument results not only in the loss of the barrier function of the skin, but also alters the perception of temperature, pain and touch. Thus, identifying an effective approach to accelerate cutaneous regeneration with the restoration of function is of paramount importance. In recent years, MSCs have gained attention in the field of cutaneous repair and regeneration. (Wu, et al.). Accordingly, the application of an exosomal composition is effective to treat burn/wounds, both chronic and acute.

Cardiovascular Disease

As discussed in the foregoing section, exosomes are a subgroup of extracellular vesicles containing a huge number of bioactive molecules. They represent an important means of cell communication, mostly between different cell populations, with the purpose of maintaining tissue homeostasis and coordinating the adaptive response to stress. This type of intercellular communication is important in the cardiovascular field, mainly because the heart is a complex multicellular system. The function of extracellular vesicles in cardiovascular and metabolic diseases shares features with its role in cancer, with emerging evidence of crosstalk between different cell types in the heart that is mediated by extracellular vesicles. For example, angiotensin II elicits the release of extracellular vesicles from cardiac fibroblasts, which can potentiate cardiac hypertrophy through altering of gene expression in cardiomyocytes. (Gray, et al). In addition, microRNA 155 that is contained within macrophage-derived extracellular vesicles decreases fibroblast proliferation and increases inflammation in mice, (Wang, et al.) which suggests that extracellular vesicle—mediated crosstalk between noncardiomyocyte-cell types may affect cardiac structure. Indeed, translational studies have borne out the concept that circulating extracellular vesicles obtained from patients with dilated cardiomyopathy can transfer a pathologic molecular phenotype to cardiomyocytes in culture, similar to what has been observed in cancer. (Deddens, et al.). In humans, the number of circulating extracellular vesicles can be increased in certain forms of cardiovascular disease (e.g., heart failure). (Jansen, et al.). Most ongoing large cohort studies have focused on metabolites, proteins, and transcriptional analyses from whole plasma, and the isolation of extracellular vesicles to quantify these biomarkers is a newer field. The concentration of circulating exosomes in plasma is proportional to circulating levels of cardiac troponin and increases at 24 to 48 hours after coronary-artery bypass surgery. (Bei, et al.) The quantity of circulating microparticles has been associated with risk factors for cardiovascular disease and with long-term cardiac prognosis (for endothelial-derived microparticles). (Franca, et al.). Furthermore, in a case—control study involving patients with and without heart failure after myocardial infarction, several microRNAs that are prognostic for heart failure and left ventricular remodeling were enriched in circulating extracellular vesicles. (Vegter, et al.).

The interrogation of protein expression within extracellular vesicles has also uncovered several proteins that are related to acute coronary disease, including an immunoglobulin receptor, cystatin C, and complement. (Boulanger, et al.). In mice with myocardial infarction, the injection of extracellular vesicles derived from cardiac progenitor cells into the periinfarct zone limited remodeling, similar to the result after the injection of progenitor cells. The benefits of extracellular vesicles may arise from effects on cardiomyocyte survival and myocardial fibrosis. (Chistiakov, et al) Similar to cardiovascular disease, cardiometabolic diseases (e.g., obesity) are also characterized by an increased number of circulating microparticles. (Da Costa, et al.). Recent studies in mouse models have shown that extracellular vesicles from adipose tissue can modulate hepatic gene expression in a manner that is dependent on noncoding RNAs, (Pomatto, et al.) which suggests a potential pathogenic role for extracellular vesicles acting at a distance in metabolic diseases. Much of the clinical data in this area remains correlative but suggests important links among diabetes, obesity, and cardiovascular disease. For example, shifts in the microRNA cargo in extracellular vesicles in patients with diabetes (e.g., reduced amounts of microRNA 126 and 26a in endothelial cell—derived microparticles) may be associated with cardiovascular disease, which potentially links the two conditions. In turn, therapies directed against dysglycemia may alter circulating extracellular vesicles. In a small study involving patients who had undergone bariatric surgery, the post-surgical shift in insulin resistance was accompanied by changes in microRNAs in extracellular vesicles that are implicated in insulin signaling. (Fernandez-Valverde, et al.). Although a great deal of work remains to trans-late early animal-based findings to humans, these preliminary findings suggest that extracellular vesicles could serve as functional biomarkers of cardiovascular and cardiometabolic diseases.

Ophthalmologic Diseases

Exosomes can significantly alleviate the symptoms of macular degeneration, faulty drainage at the trabecular angle with respect to glaucoma and dry eye damage.

Neurological Diseases

The strong paracrine capacity of exosome and not their differentiation capacity, is the principal mechanism of therapeutic action. MSCs robustly release exosomes, membrane vesicles (˜30-100 nm) originally derived in endosomes as intraluminal vesicles, which contain various molecular constituents including proteins and RNAs from maternal cells. Contained among these constituents, are small non-coding RNA molecules, microRNAs (miRNAs), which play a key role in mediating biological function due to their prominent role in gene regulation. The release as well as the content of the MSC generated exosomes are modified by environmental conditions. Via exosomes, MSCs transfer their therapeutic factors, especially miRNAs, to recipient cells, and therein alter gene expression and thereby promote therapeutic response. The packaging and transfer of miRNAs which enhance tissue repair and functional recovery in stroke.

Neutrophic factors in exosomes possess regenerative powers that could treat neurological illnesses including Parkinsons.

In the last decades, exosomes were also shown to facilitate cell-to-cell transport of disease-related proteins involved in neurodegenerative disorders, such as prions (Alenquer, et al.) and beta-amyloid peptides. (Sinha, et al.). This machinery could, in principle, also contribute to viral spread. For this to happen, two prerequisites would be necessary. First, viral RNA and proteins would need to access ILV. Indeed, vesicular stomatitis virus (VSV), dengue virus (and other Flavivirus members), and hepatitis C virus (HCV) components were found in these sub-compartments. (Alenquer, et al.).

With the emergence of similar themes in cancer, cardiovascular disease, and neurologic disease, researchers have intense interest in exploring the potential role of extracellular vesicles in neurodegeneration, trauma, and stroke. In models of traumatic brain injury, the presence of increased amounts of microRNA in extracellular vesicles from microglia has been associated with decreased inflammation and improved regrowth after injury. Similarly, in models of stroke, micro-RNA 133b contained within extracellular vesicles from stromal cells may be involved in improvements in neural structure. The notion that extracellular vesicles may be involved in transfer-ring phenotypes between diseased and healthy tissues, as has been observed in patients with cancer and cardiovascular disease, may also apply to neurocognitive diseases (e.g., Lewy body dementia). Emerging studies suggest a role for extracellular vesicle cargo in neurocognitive diseases. Phosphorylated tau protein that is associated with extracellular vesicles in cerebrospinal fluid appears early in Alzheimer's disease, and secretion of extracellular vesicles containing tau may be mechanistically important in Alzheimer's dis-ease. Altered expression of key proteins that are directly involved in synaptic physiologic function can be found in extracellular vesicles from plasma in direct proportion to cognitive dysfunction in adults with Alzheimer's disease, and select extracellular-vesicle protein contents in circulating blood can indicate a high risk of Alzheimer's disease several years before clinical diagnosis. Next-generation RNA sequencing and polymerase-chain-reaction assay of extracellular vesicles from serum have identified a panel of 16 microRNAs that are dysregulated in Alzheimer's disease. In addition, specific microRNA contents in extracellular vesicles from cerebrospinal fluid may be distinct in different neurocognitive diseases (e.g., Parkinson's and Alzheimer's disease), findings that may be applicable to diagnosis if they are verified in large studies. In one study, circulating levels of extracellular vesicles containing tau protein were higher in players in the National Football League than in healthy controls and were related to poorer neurocognitive performance in football players, findings that are consistent with an emerging recognition of chronic traumatic encephalopathy as a related disease entity. These results are especially intriguing in light of the emergence of point-of-care detection of circulating neuronal-cell—derived exosomes that are present after concussive injury in mice. Similar to approaches in cancer, extra-cellular vesicle—based therapies that target neuro-degenerative disease are rapidly emerging—for example, engineering of extracellular vesicles containing small interfering RNAs that alter the expression of an enzyme involved in the generation of beta-amyloid deposits.

Cancers

The role of extracellular vesicles and their contents as potential contributors to oncogenesis, metastatic disease, and resistance to chemotherapy is a rapidly expanding area of research in cancer biology. Extracellular vesicles can funnel chemotherapeutic agents out of a cancer cell through bulk transport within vesicles or active efflux mechanisms and may also express molecules that divert biologic agents away from malignant cells (e.g., human epidermal growth factor receptor 2 [HER2] in breast cancer). In ovarian cancer, interactions between stromal tissue and cancer cells that are mediated by extracellular vesicles may transfer microRNA 21, which can potentiate resistance to chemotherapy. Extracellular vesicles may also be involved in metastasis by harboring molecules that are involved in the epithelial—mesenchymal transition or preparing target tissues for metastasis. Furthermore, studies of breast cancer—derived exosomes suggest that they contain proteins required for microRNA-mediated gene silencing and may transform nonmalignant cells. Finally, on the basis of the premise that extracellular vesicles bearing antigens derived from cancer cells may originate from the parent cancer cells, investigators have isolated extracellular vesicles from the plasma of patients with acute myeloid leukemia to evaluate whether these vesicles may alter the expression of molecules important in immune-cell function. These findings implicate extra-cellular vesicles in different steps of carcinogenesis and therapeutic responses and suggest that they may play a functional role in specific aspects of cancer. Studies enrolling human participants have started to illuminate a role for extracellular vesicles in diagnosis, prognosis, and therapy in cancer. In ovarian cancer, the quantity of circulating extracellular vesicles that presumably originated from tumor tissue (on the basis of a cell-surface marker) was proportional to the cancer stage and was greater than the quantity in healthy controls. In addition, extracellular vesicles and their cargo have been explored as part of a diagnostic or prognostic strategy for various cancers, including cancers of the hepatobiliary system, breast, lung, gastrointestinal tract, skin (melanoma), prostate, and nasopharynx. Efforts have focused on the discovery of biomarkers in extracellular vesicles across multiple biofluids relevant to each cancer, including proteins in circulating blood for colorectal cancer, urinary microRNAs for prostate cancer and proteins for bladder cancer, and microRNA profiles in cerebrospinal fluid for brain cancer. Specific molecules in extracellular vesicles have also been linked to diagnosis and staging (e.g., microRNA 21 for esophageal cancer). In light of this potential to affect the pathophysiological processes in cancer development, extracellular vesicles are increasingly being investigated as part of a new mode of cancer treatment. Specific ongoing efforts include the use of extracellular vesicles to mediate anticancer immunity and to serve as vectors for small-molecule delivery. Further basic and clinical investigations of the specificity and off-target effects of extracellular vesicles are needed before such uses can be adopted in clinical practice.

Viral Diseases

Exosomes containing viral genomes can promote viral spread by infecting adjacent, or in some cases distant permissive cells, while evading immune recognition, thanks to the absence of viral glycoproteins on the exosome membrane. Conversely, exosomes containing viral proteins or nucleic acids have been found to activate immune responses in myeloid cells in certain cases. Antigen-loaded dendritic cells can activate T cells by directly transferring exosomes to an interacting T cell, although some viruses, like HIV, have evolved to utilize DC to T-cell vesicle transfer as a route for productive infection.

Important human pathogens such as the human immunodeficiency virus (HIV), the Ebola virus, the rabies virus, and the herpes simplex virus 1 (HSV1) all have well-characterized strategies to hijack members of the endosomal sorting complexes required for the transport (ESCRT) pathway. The ESCRT pathway is the best understood mechanism underlying ILV biogenesis. However, there are viruses that manage to bud from cells via ESCRT-independent pathways. Classical examples include the influenza A virus (IAV), the severe acute respiratory syndrome Corona virus, alphaviruses like chikungunya, and pneumoviruses like respiratory syncytial virus (RSV). Exosomes released from cells infected with a variety of animal viruses, including viral spread, host immunity, and manipulation of the microenvironment. Given the ever-growing roles and importance of exosomes in viral infections, understanding what regulates their composition and levels, and defining their functions is imperative.

Exosomes are extracellular vesicles released upon fusion of multivesicular bodies (MVBs) with the cellular plasma membrane. They originate as intraluminal vesicles (ILVs) during the process of MVB formation. Exosomes were shown to contain selectively sorted functional proteins, lipids, and RNAs, mediating cell-to-cell communications and hence playing a role in the physiology of the healthy and diseased organism. Challenges in the field include the identification of mechanisms sustaining packaging of membrane-bound and soluble material to these vesicles and the understanding of the underlying processes directing MVBs for degradation or fusion with the plasma membrane. The investigation into the formation and roles of exosomes in viral infection is in its early years. Although still controversial, exosomes can, in principle, incorporate any functional factor, provided they have an appropriate sorting signal, and thus are prone to viral exploitation. Exosomes released from cells infected with a variety of animal viruses could various cellular effects including viral spread, host immunity, and manipulation of the microenvironment. Given the ever-growing roles and importance of exosomes in viral infections, understanding what regulates their composition and levels, and defining their functions will ultimately provide additional insights into the virulence and persistence of infections.

In an embodiment, the exosomes are derived from mammalian cells, and in some embodiment, the cells are human, including embryonic stem cells, pluripotent stem cells, induced pluripotent stem cells, mesenchymal stem cells, and hematopoietic stem cells among others. In an embodiment, the cells are autologous to the individual and in other embodiments, the cells are allogeneic to the individual. Xenogeneic or syngeneic cells are utilized in other embodiments.

One of the main functions assigned to exosomes is the mediation of intercellular communication during innate and adaptive immune responses. In fact, many different cells of the immune system, including dendritic cells and B and T lymphocytes, have been shown to release exosome vesicles with immune modulatory properties. These exosomes can be found in bodily fluids.

In 1996, Raposo et al. demonstrated that B lymphocytes infected with Epstein-Barr virus (EBV), a human gammaherpes virus associated with a variety of lymphoblastoid and epithelial cancers, released exosomes containing MHC II molecules, and that these vesicles were capable of activating specific CD4⁺ T cell clones in vitro. Two years later, Zitvogel et al. published a study showing that exosomes released by dendritic cells had the ability to suppress the growth of tumors in vivo. This led to the interpretation that exosomes could be used as therapeutic agents modulating immune responses.

It was recently reported that exosomes also regulate innate immunity. This was illustrated with the identification of important innate immune effectors (IFI16, caspase-1, interleukin 1b (IL-1b), IL-18, and IL-33) in exosomes released from EBV-infected cells. Such a strategy removes these effectors from infected cells to reduce innate immunity activation. Another interesting approach was proposed for HSV1 infection. In this case, cells export the innate immune sensor STING (stimulator of IFN genes), viral miRNAs, and mRNAs through exosomes that are delivered to uninfected cells. The functional significance of this strategy is still not clear, but the fact that some miRNAs are able to suppress reactivation of latent virus suggests that, in specific circumstances, HSV1 has evolved mechanisms to restrict, rather than expand, the spread of infection. Exosomes harboring HCV RNA are transferred from infected cells to non-permissive plasmacytoid DCs, where viral RNA can trigger a type I IFN response.

It is clear that in viral infections, exosomes play a dual role in the modulation of the immune system, both serving as a host program to induce innate and adaptive immunity and as a viral strategy to evade those same responses.

Viruses can harness the cellular machinery of extracellular vesicles for multiple purposes, including increasing infectivity and evading the immune system. Extracellular vesicles that are derived from hepatoma cells infected with hepatitis C in vitro contain genetic information and proteins that promote infection in the absence of active interaction between viruses and target cells. Moreover, this extracellular vesicle—mediated infection may elude antibody-mediated immune clearance.64 From a biomarker perspective, the morphologic features and quantity of extracellular vesicles appear to correspond to the activity of viral infection. In patients who are infected with the human immunodeficiency virus type 1 (HIV-1), both the size and quantity of circulating extra-cellular vesicles are inversely proportional to the ratio of CD4 to CD8 T cells, with a smaller size and lower quantity associated with a higher CD4:CD8 ratio (indicating strong immune function). The control of HIV-1 infection is associated with near normalization of the morphologic features of extracellular vesicles.65 Furthermore, treatment with antiretroviral therapy is associated with a reduced amount of microRNA 155 (a pro-inflammatory noncoding RNA) and of microRNA 223 in extracellular vesicles, which suggests that the contents of extracellular vesicles may provide a molecular signature of response to therapy.65 The infectious potential of extracellular vesicles extends to prion disease,66 in which extracellular vesicles bearing prion protein that has been converted into its pathogenic insoluble conformer (PrPSc) may transmit the disease.67 Further studies of extracellular vesicles as biomarkers for therapy, early detection, or tracking of infection are warranted.

Combination Therapies

The methods described herein can further comprise administering one or more additional therapeutic agents. The one or more additional therapeutic agents can be administered to a patient simultaneously or sequentially.

In some embodiments, the additional therapeutic agent is an antibiotic. In some embodiments, the antibiotic is clindamycin, doxycycline, minocycline, trimethoprim-sulfamethoxazole, erythromycin, metronidazole, rifampin, moxifloxacin, dapsone, or a combination thereof. In some embodiments, the antibiotic is clindamycin, doxycycline, minocycline, trimethoprim-sulfamethoxazole, or erythromycin in combination with metronidazole. In some embodiments, the antibiotic is a combination of rifampin, moxifloxacin, and metronidazole. In some embodiments, the antibiotic is a combination of moxifloxacin and rifampin.

In some embodiments, the additional therapeutic agent is a retinoid. In some embodiments, the retinoid is etretinate, acitretin, isotretinoin, or a combination thereof.

In some embodiments, the additional therapeutic agent is a steroid. In some embodiments, the additional therapeutic agent is a coriticosteroid. In some embodiments, the steroid is triamcinolone, dexamethasone, fluocinolone, cortisone, prednisone, prednisolone, flumetholone, or a combination thereof.

In some embodiments, the additional therapeutic agent is an immunosuppressant. In some embodiments, the immunosuppressant is methotrexate, cyclosporin A, or a combination thereof. In some embodiments, the immunosuppressant is mycophenolate mofetil, mycophenolate sodium, or a combination thereof.

In some embodiments, the additional therapeutic agent is finasteride, metformin, adapalene, azelaic acid, or a combination thereof.

In some embodiments, the additional therapeutic agent is an anti-angiogenic agent, a cholinergic agonist, a TRP-1 receptor modulator, a calcium channel blocker, a mucin secretagogue, a MUC1 stimulant, a calcineurin inhibitor, a corticosteroid, a P2Y2 receptor agonist, a muscarinic receptor agonist, an mTOR inhibitor, a JAK inhibitor, a Bcr-Abl kinase inhibitor, a Flt-3 kinase inhibitor, a RAF kinase inhibitor, a FAK kinase inhibitor, or a combination thereof. In some embodiments, the additional therapeutic agent is a tetracycline derivative (e.g., minocycline or doxycline). In some embodiments, the additional therapeutic agent binds to FKBP12.

In some embodiments, the additional therapeutic agent is an alkylating agent or a DNA cross-linking agent; an anti-metabolite/demethylating agent (e.g., 5-flurouracil, capecitabine or azacitidine); an anti-hormone therapy (e.g., hormone receptor antagonists, SERMs, or aromotase inhibitor); a mitotic inhibitor (e.g. vincristine or paclitaxel); an topoisomerase (I or II) inhibitor (e.g. mitoxantrone and irinotecan); an apoptotic inducers (e.g. ABT-737); a nucleic acid therapy (e.g. antisense or RNAi); a nuclear receptor ligand (e.g., agonist and/or antagonist: all-trans retinoic acid or bexarotene); an epigenetic targeting agent such as a histone deacetylase inhibitor (e.g. vorinostat), a hypomethylating agent (e.g. decitabine); a regulator of protein stability such as a Hsp90 inhibitor, an ubiquitin and/or ubiquitin like conjugating or deconjugating molecules; or an EGFR inhibitor (erlotinib); or a combination thereof.

In some embodiments, the additional therapeutic agent includes an antibiotic, an antiviral, an antifungal, an anesthetic, or an anti-inflammatory agent including steroidal and non-steroidal anti-inflammatory agent, or anti-allergic agent, or a combination thereof. Examples of suitable medicaments include an aminoglycoside such as amikacin, gentamycin, tobramycin, streptomycin, netilmycin, and kanamycin; fluoroquinolone such as ciprofloxacin, norfloxacin, ofloxacin, trovafloxacin, lomefloxacin, levofloxacin, and enoxacin; naphthyridine; sulfonamide; polymyxin; chloramphenicol; neomycin; paramomycin; colistimethate; bacitracin; vancomycin; tetracyclines; rifampin and its derivatives (“rifampins”); cycloserine; beta-lactam; cephalosporin; amphotericin; fluconazole; flucytosine; natamycin; miconazole; ketoconazole; corticosteroid diclofenac; flurbiprofen; ketorolac; suprofen; cromolyn; lodoxamide; levocabastin; naphazoline; antazoline; pheniramine; or azalide antibiotic, or a combination of all the above.

Pharmaceutical Formulations and Dosage Forms

When employed as a therapeutic, the compound of the invention can be administered in the form of a pharmaceutical composition. The composition can be prepared in a manner well known in the pharmaceutical art, and can be administered by a variety of routes, depending upon whether local or systemic treatment is desired and whether a particular area is to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or by a continuous perfusion pump. In an embodiment, a pharmaceutical compositions or formulations for topical administration may include a transdermal patch, an ointment, a lotion, a cream, a gel, a drop, a suppository, a spray, a liquid and a powder. Any conventional pharmaceutical carrier, an aqueous, a powder or an oily base, a thickener and the like may be necessary or desirable.

In some embodiments, the administration is topical. In some embodiments, the administration is a topical administration to the skin.

In preparing a formulation, the active compound can be milled to provide the appropriate particle size prior to combining with the other ingredients. If the active compound is substantially insoluble, it can be milled to a particle size of less than about 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: a lubricating agent such as talc, magnesium stearate, and mineral oil; a wetting agent; an emulsifying and a suspending agent; a preserving agent such as methyl- and propylhydroxy-benzoate; a sweetening agent; and a flavoring agent. The composition of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, a wet granulation process is used to produce the composition. In some embodiments, a dry granulation process is used to produce the composition.

The compositions can be formulated in a unit dosage form, each dosage containing from about 1 to about 1,000 mg, from about 1 mg to about 100 mg, from 1 mg to about 50 mg, and from about 1 mg to 10 mg of active ingredient. Preferably, the dosage is from about 1 mg to about 50 mg or about 1 mg to about 10 mg of active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions comprise from about 1 to about 1,000 mg, from about 1 mg to about 100 mg, from 1 mg to about 50 mg, and from about 1 mg to 10 mg of active ingredient. Preferably, the compositions comprise from about 1 mg to about 50 mg or about 1 mg to about 10 mg of active ingredient. One having ordinary skill in the art will appreciate that this embodies compounds or compositions containing about 1 mg to about 10 mg, about 1 mg to about 20 mg, about 1 mg to about 25 mg, about 1 mg to about 50 mg of the active ingredient.

In some embodiments, the dosage of the compound, or a pharmaceutically acceptable salt thereof, is about 15, 30, 60 or 90 mg on a free base basis. In some embodiments, the dosage is about 15, 30, 60 or 90 mg on a free base basis, of Compound 4, or a pharmaceutically acceptable salt thereof. In some embodiments, the dosage of the compound, or a pharmaceutically acceptable salt thereof, is about 15 mg on a free base basis. In some embodiments, the dosage of the compound, or a pharmaceutically acceptable salt thereof, is about 30 mg on a free base basis. In some embodiments, the dosage of the compound, or a pharmaceutically acceptable salt thereof, is about 60 mg on a free base basis. In some embodiments, the dosage of the compound, or a pharmaceutically acceptable salt thereof, is about 90 mg on a free base basis.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

The liquid form in which the compounds and composition of the present application can be incorporated for administration by injection includes aqueous solution, suitably flavored syrup, aqueous or oil suspension, and flavored emulsion with edible oil such as cottonseed oil, sesame oil, coconut oil, or peanut oil, as well as any elixir and similar pharmaceutical vehicle.

In an embodiment, a composition for inhalation or insufflation includes a solution and/or a suspension in pharmaceutically acceptable, aqueous or organic solvent, or mixture thereof, and powders. The liquid or solid compositions may contain suitable pharmaceutically acceptable excipients as described supra. In some embodiments, the compositions are administered by the oral or nasal respiratory route for local or systemic effect. In an embodiment, a composition can be nebulized by using inert gases. Nebulized solutions may be breathed directly from a nebulizing device or a nebulizing device can be attached to a face mask tent, or an intermittent positive pressure breathing machine. Solution, suspension, or powder compositions can be administered orally or nasally from devices which deliver the formulation in an appropriate manner.

In an embodiment, a topical formulation can contain one or more conventional carriers. In some embodiments, ointments can contain water and one or more hydrophobic carriers selected from, for example, liquid paraffin, polyoxyethylene alkyl ether, propylene glycol, white Vaseline, and the like. A carrier composition of cream can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, a topical formulation contains at least 5 about 0.1, at least about 0.25, at least about 0.5, at least about 1, at least about 2, or at least about 5 wt % of the compound of the invention. The topical formulation can be suitably packaged in tubes of, for example, about 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In a therapeutic application, a composition can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The composition administered to a patient can be in the form of a pharmaceutical composition as described above. The composition can be sterilized by a conventional sterilization technique, or it may be sterile filtered. Aqueous solution can be packaged for use as is, or lyophilized, the lyophilized preparation can be combined with a sterile aqueous carrier prior to administration. The pH of the compound preparations typically will be between about 3 and about 11, more preferably from about 5 to about 9 and most preferably from about 7 to about 8. It will be understood that use of certain of any of the foregoing excipients, carriers, or stabilizers will result in the formation of pharmaceutical salts.

The therapeutic dosage of a compound of the present application can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, a compound of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents, examples of which are listed hereinabove.

Scaffolding

The scaffolding serves two purposes. First is the differentiation of stem cells, including MSCs, in a 3D environment or configuration into specific lines. Another purpose is the facilitation of therapy, regeneration and healing in various integumentary and bone tissues. The combination of exosome products with biologically active scaffold materials for wound care/skin regeneration, bone regeneration. The scaffold includes nanofibrous polymers, hyaluronic acid, chitosan, hydrogel, laminin, collagen, and alginate, among other molecules. Scaffolding with NO and chitosan tend to increase release of exosomes from placental MSCs. These MSCs are pro-angiogenic, pro-migratory with resulting increase in VEGF and miR126 with ischemic muscle tissue protected.

In an embodiment, the purification and isolation of exosomes and/or exosomal products comprise the following four major steps: (1) Recovery of MSC; (2) Processing of MSC using Micelle technology; (3) Filtration/separation steps; and (4) Purification and sterilization.

In the first general step, MSCs are recovered from perinatal tissue, 3D or 2D cell cultures. MSCs grown from 2D cell cultures are imparted unique characteristics to the MSC and their exosome through interaction with scaffold materials. For example, if the scaffolding is high in chitosan and hyaluronic acid, the bias for the MSC is toward osteogenesis. Alternatively, the addition of nitric oxide (NO)+chitosan increases the release of exosomes from placental MSC. These MSCs are pro-angiogenic and pro-migratory resulting in increased vasoendothelial growth factor (VEGF) and increased miR126 with ischemic muscle tissue protected.

The MSCs are then processed using Micelle technology. An illustrative Micelle used is poloxomer 188/407. Other similar Micelle can be utilized.

The MSCs are then isolated through filtration by centrifugation and size exclusion filtration technology.

The MSCs are then purified and sterilized resulting in highly consistent exosomes with biological active exosomal cargo/products.

A person of ordinary skill under the art can appreciate other types of stem cells or from other sources can be used and general variations in the techniques can be utilized.

### EXAMPLES

The invention will be described in greater detail by way of specific examples. The following examples are offered for illustrative purposes, and they are not intended to limit the invention in any manner. Those of skill in the art will readily recognize a variety of non-critical parameters which can be changed or modified to yield essentially the same results.

### Example A: Isolation of Exosomes from Wharton's Jelly

Wharton's jelly is a gelatinous substance within the umbilical cord, largely made up of mucopolysaccharides. It acts as a mucous connective tissue containing some fibroblasts and macrophages, and it is derived from extra-embryonic mesoderm. Cells in Wharton's jelly express several stem cell genes, including telomerase. They can be extracted, cultured, and induced to differentiate into mature cell types such as neurons. (Citation) Wharton's jelly is therefore a potential source of adult stem cells, often collected from cord blood.

Wharton's Jelly was initially processed as outlined in Table X to prepare for exosome isolation and purification.

TABLE 2
Processing Wharton's Jelly

| Step | Description |
|---|---|
| 1 | Add 100_mL 0.9% Sodium Chloride Irrigation Solution (Normal Saline) |
| | into a sterile cup. |
| 2 | Using a sterile scalpel and forceps to cut the umbilical cord tissue |
| | into approximately 1 inch segments. |
| 3 | Make a deep longitudinal incision on segment and cord open to expose |
| | blood vessel. Perform this for each segment of umbilical cord. |
| 4 | Remove and discard the blood vessel. |
| 5 | Using Sterile forceps, rinse the umbilical cord segment with 100_mL of |
| | 0.9% normal saline into a sterile cup. Repeat for each umbilical cord |
| | segment. Swirl contents gently to remove excess blood. |
| 6 | Transfer the umbilical cord tissue into a sterile petri dish and add 10 mL of |
| | PBS. |
| 7 | Allow petri dish with umbilical cord and 10 mL of PBS to sit for 30 |
| | minutes under UV light. |
| 8 | Record the start and end times. |
| 9 | Place Wharton's Jelly supernatant/body fluid in 50 mL conical tubes for |
| | further processing. |

Following the initial processing of Wharton's Jelly, the tissue and fluids were processed as outlined in Table 3 to isolate exosomes.

TABLE 3
Exosome Isolation from Wharton's Jelly

| Step | Description |
|---|---|
| 1 | Start with Wharton's Jelly supernatant/body fluid in 50 mL conical tubes. |
| 2 | Transfer approximately 6 grams of tissue to 1 50 mL centrifuge tubes. |
| 3 | Add 22 mL of PBS (0.33 cc/gram of tissue) added to conical tubes. |
| 4 | Vortex for 1 minute. |
| 5 | Spin at 100 × g for 10 minutes. |
| 6 | Discard pellet and place the supernatant into sterile conical tube. |
| 7 | Place all supernatant from STEP 6 into sterile beaker. |
| 8 | Place 20 mL or equal amounts of supernatant into individual 50 mL centrifuge |
| | tubes. |
| 9 | Spin at 300 × g for 10 minutes. |
| 10 | Discard pellet and place supernatant without disturbing pellet into new sterile |
| | conical tube. |
| 11 | Collect supernatant from STEP 10. |
| 12 | Aliquot equal amounts of supernatant in new 50_mL centrifuge tubes. |
| 13 | Spin at 5,000 × g for 10 minutes. |
| 14 | Discard pellet and place supernatant into another sterile conical tube. |
| 15 | Collect supernatant from STEP 14. |
| 16 | Spin at 100,000 × g for 70 minutes. |
| 17 | KEEP PELLET (pellet contains exosomes and proteins). |
| 18 | Remove 17 mL of supernatant and replace with 17 mL of PBS. |
| 19 | Collect pellet from STEP 17. |
| 20 | Wash with PBS-Remaining pellet contains exosomes. |
| 21 | Wash/dilute pellet into total volume of 240 mL in a 250 mL glass beaker on a |
| | magnetic stirrer at room temperature. |
| 22 | Aliquot 20 mL into each of 12 conical tubes. |
| 23 | Spin at 100,000 × g for 70 minutes. |

### Example B: Processing of Peripheral Blood for Exosome Isolation

Peripheral blood is collected from venous puncture and provides a potential source of exosomes as well. Peripheral blood was collected from donors and processed as outlined in Table 4.

TABLE 4
Peripheral Blood Processing for Exosome Isolation

| | |
|---|---|
| Step | Description |

Dilution of Blood

| | |
|---|---|
| 1 | Obtain blood samples that were sent from clinical site. |
| 2 | Pool blood of each patient from Vacutainers into 250 ml Corning Plastic |
| | Storage Bottle. |
| 3 | Dilute blood with an equal amount of 1X DPBS (room temperature). Mix |
| | gently. |

Isolation of PBMC's by Ficoll Density Gradient Centrifugation

| | |
|---|---|
| 4 | Add 15 ml room temperature Ficoll to a sterile 50 ml conical tube. |
| 5 | Gently overlay the Ficoll with 30 ml of diluted blood (from Step 5.1.3) |
| | using a sterile serological pipette. Minimize the mixing of the two |
| | phases. |
| 6 | Centrifuge at 2000 rpm for 30 minutes at room temperature with brake |
| | off to ensure that deceleration does not disrupt the density gradient. |
| 7 | After the centrifuge stops, carefully remove the tube without disturbing |
| | the pellet. Using a pipette, carefully aspirate the diluted plasma layer and |
| | pipette into separate 15 mL conical for further exosome isolation as |
| | outlined in EXAMPLE C. DO NOT DISTURB the lymphocyte and |
| | monocyte band (PBMC layer). Collect the PBMCs (cloudy layer) from |
| | the diluted plasma/Ficoll interface using a sterile serological pipette and |
| | place the cells into a sterile 50 ml conical tube. |
| | Interface cells from a maximum of two 50 ml tubes can be combined into |
| | one wash tube. |
| | IMPORTANT: While collecting the cells, be sure to aspirate as little |
| | Ficoll as possible. Lower cell numbers will pellet if the proportion of |
| | Ficoll is too high in the wash tube. |
| 8 | Add 1x DPBS to bring the volume up to 45 ml. Gently pipette up and |
| | down to mix cell solution with DPBS. |
| 9 | Centrifuge at 1500 rpm for 10 minutes at RT. Keep brake on - low. |
| 10 | After the centrifuge stops, carefully remove the tube without disturbing |
| | the pellet. Using a serological pipette, carefully remove and discard the |
| | supernatant without touching the pellet. |
| | Note: |
| | After most of the supernatant is aspirated, remove remaining |
| | supernatant by progressively tilting the tube while the pipette tip touches |
| | the side of the tube. Do not lower pipette tip close to the cell pellet. |
| 11 | Loosen the pellet by tapping the tube with your finger. Then add 5 ml of |
| | sterile DPBS and carefully mix the cells by gently pipetting up and |
| | down. |
| 12 | Pool all the individual cell suspensions into a single sterile 50 ml tube. |
| 13 | Bring the total volume of the cell suspension to 40 ml with DPBS |
| | washing solution |
| 14 | Mix cells well by pipetting. Take out a 25 μl aliquot from the cell |
| | suspension to count cells. |

Magnetic Labeling of Lin+ Cells

| | |
|---|---|
| 24 | Centrifuge cell suspension at 300 × g for 10 minutes. Aspirate supernatant |
| | completely. |
| 25 | Resuspend cell pellet in 200 μL of MACS buffer (see Appendix B for |
| | preparation) per 5 × 10⁷ total cells. |
| 26 | Add 50 μL of CD34 Diamond MicroBeads per 5 × 10⁷ total cells. |
| 27 | Mix well and incubate for 30 minutes in the refrigerator (2-8° C.). |
| 28 | (Optional) Add staining antibodies and incubate for 10 minutes in the |
| | dark in the refrigerator (2-8° C.). |
| 29 | Wash cells by adding 2.5-5 mL of MACS buffer (see Appendix B for |
| | preparation) per 5 × 10⁷ cells and centrifuge at 300 × g for 10 minutes. |
| | Aspirate supernatant completely |
| 30 | Resuspend cell pellet in 500 μL of MACS buffer per 10⁸ total cells. |
| 31 | Transfer cells to 50 mL centrifuge tube |
| 32 | Isolate exosomes from cell pellets as described in EXAMPLE A |

### Example C: Isolation of Exosomes from Plasma

Using plasma from peripheral blood, exosomes were purified as outlined in Table 5.

TABLE 5
Exosome Isolation from Plasma

| | |
|---|---|
| Step | Description |

Dilution of Blood

| | |
|---|---|
| 1 | Remove the plasma sample from storage and place on ice. If the sample is |
| | frozen, thaw the sample in a 25° C. to 37° C. water bath until it is completely |
| | liquid, and place on ice until needed. |
| 2 | Centrifuge the plasma sample at 2000 × g for 20 minutes at room temperature |
| | to remove cells and debris. |
| 3 | Transfer the supernatant containing the partially clarified plasma to a new tube |
| | without disturbing the pellet. |
| 4 | Centrifuge the new tube at 10,000 × g for 20 minutes at room temperature to |
| | remove debris. |
| 5 | Transfer the supernatant containing the clarified plasma to a new tube without |
| | disturbing the pellet, and place it on ice until ready to perform the isolation |

Exosome Isolation

| 6 | Transfer the required volume of clarified plasma to a new tube and add 0.5 | |
|---|---|---|
| | volumes of 1X PBS. | |
| 7 | Mix the sample thoroughly by vortexing. | |
| 8 | Add 0.2 volume (i.e. Total volume = plasma + PBS) of the Exosome | |
| | Precipitation Reagent (from plasma) to the sample. | |
| | Plasma + PBS | Reagent |
| | 100 μL + 50 μL | 30 μL |
| | 1 mL + 0.5 mL | 300 μL |

| | |
|---|---|
| 9 | Mix the plasma/reagent mixture well either by vortexing or inversion until the |
| | solution is homogenous. |
| | Note: |
| | The solution should have a cloudy appearance. |
| 10 | Incubate the sample at room temperature for 10 minutes. |
| 11 | After incubation, centrifuge the sample at 10,000 × g for 5 minutes at room |
| | temperature. |
| 12 | Aspirate the supernatant by pipetting and discard. |
| | Note: |
| | Exosomes are contained in a pellet at the bottom of the tube. |
| 13 | (Optional) Centrifuge the tube for 30 seconds at 10,000 × g to collect any |
| | residual reagent. |
| 14 | Discard any residual supernatant by careful aspiration with a pipet and |
| | proceed to “Resuspend Exosomes.” |

Exosome Suspension

| 15 | Add 1X PBS or similar buffer to the pellet and vortex or pipet up and down to | |
|---|---|---|
| | resuspend the exosomes. | |
| | Starting Plasma Volume | Resuspension Volume |
| | 100 μL | 25-50 μL |
| | 1 mL | 100-500 μL |

| | |
|---|---|
| 16 | Once the pellet is resuspended, the exosomes are ready for downstream |
| | analysis or further purification through affinity methods |
| 17 | Keep isolated exosomes at 2° C. to 8° C. for up to 1 week, or at −20° C. or colder |
| | for long-term storage. |

### Example D: Characterization of Exosomes

Exosomes isolated in Example A were further characterized by assessing criteria outlined in Table 6.

TABLE 6
Exosome Characterization

| Assessment | Description |
|---|---|
| Micro RNA | miR21 |
| | miR 146a |
| | miR 181c |
| | miR 124a |
| | miR 125b |
| | miR 21 |
| | miR 23a |
| | miR 125b |
| | miR 145 |
| | miR 451 |
| | miR 133b |
| | miR 126 |
| | miR 296 |
| Proteins | Annexin A1 |
| | B7-2 |
| | Clathrin |
| | Flotillin-1 |
| | MFG-E8 |
| | MHC I |
| | MHC II |
| | Tsg 1 |
| | Tetraspanins |
| | Neprilysin |
| Cellular Markers | CD9 |
| Cluster of | CD34 |
| differentiation | CD41 |
| (CD) | CD45 |
| | CD59 |
| | CD63 |
| | CD73 |
| | CD81 |
| | CD105 |
| | CD117 |
| | CD123 |
| | CD146 |
| | CD166 |
| Sizing | 50 to 100 nm |
| | 20 to 200 nm |

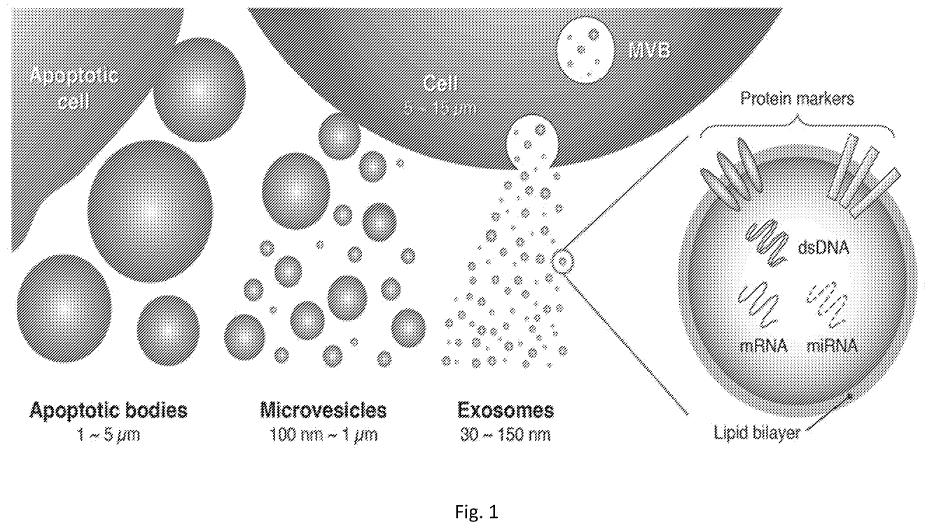

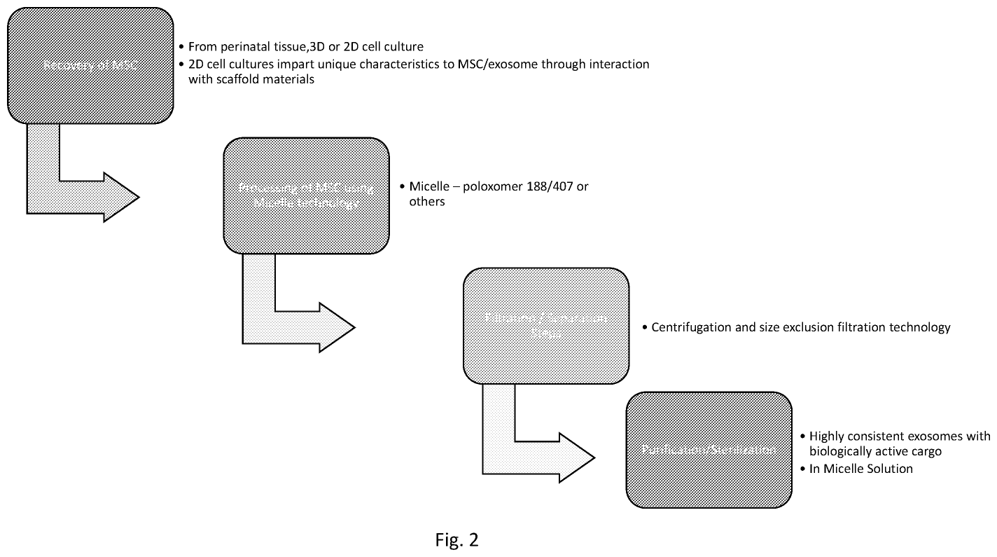

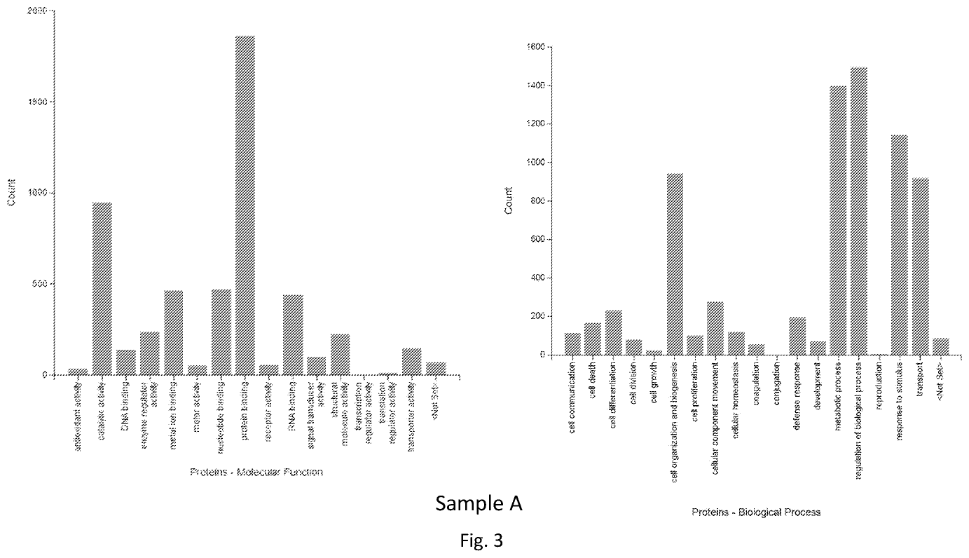

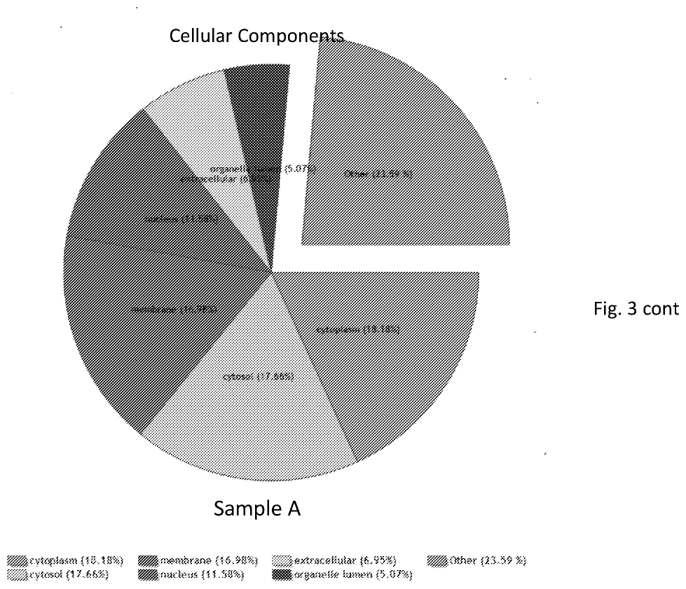

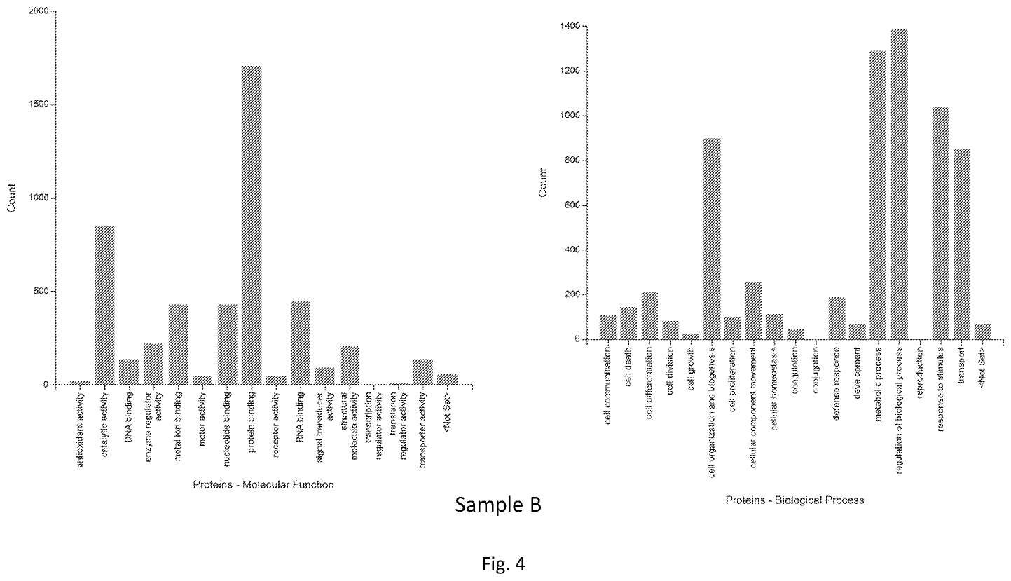

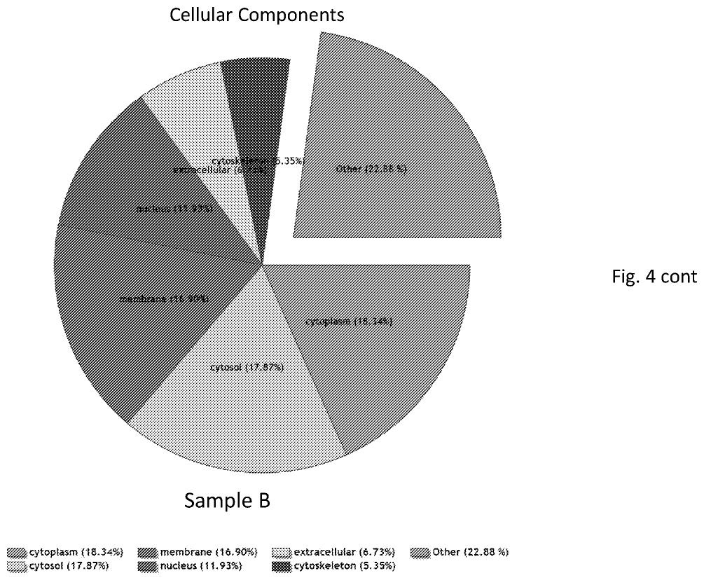

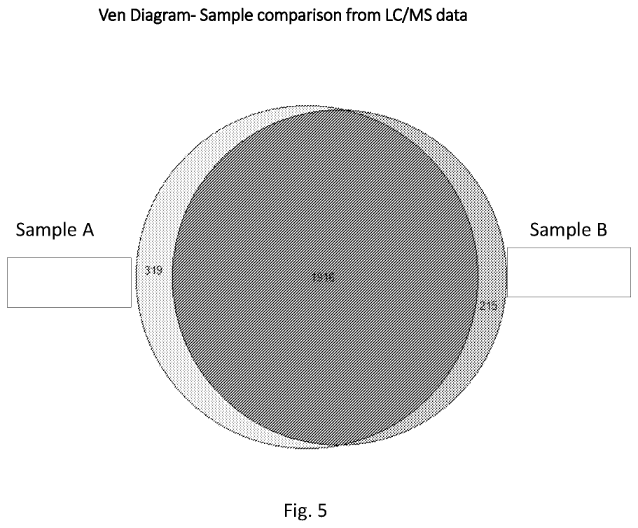

Table 7 outlines average percent and range for each of the cellular markers and FIG. 1 illustrates the percentage of exosomes which are positive or negative for specific cellular markers.

TABLE 7
Average Percent and Range for Cellular Markers on Exosomes

| | Average % of | Average % of |
|---|---|---|
| | Exosome Positive* | Exosome Negative* |
| Marker | [Range] | [Range] |
| CD9 | 71.2 ± 1.5 | 28.8 ± 1.5 |
| | [69.0-74.0] | [26.0-31.0] |
| CD59 | 40.0 ± 7.2 | 60.0 ± 7.2 |
| | [28.1-53.1] | [46.9-71.9] |
| CD63 | 15.7 ± 1.7 | 84.3 ± 1.7 |
| | [12.3-17.6] | [82.4-87.7] |
| CD73 | 73.2 ± 5.9 | 26.8 ± 5.9 |
| | [62.7-83.0] | [17.0-37.3] |
| CD81 | 73.3 ± 6.2 | 26.7 ± 6.2 |
| | [62.2-83.5] | [16.5-37.8] |

Various modifications of the invention, in addition to those described herein, will be apparent to those skilled in the art from the foregoing description. Such modifications are also intended to fall within the scope of the appended claims. Each reference cited in the present application, including all patent, patent applications, and publications, is incorporated herein by reference in its entirety.

### REFERENCES

Ankrum J A, Ong J F, Karp J M (March 2014). Mesenchymal stem cells: immune evasive, not immune privileged. Nature Biotechnology. 32 (3): 252-60.
Bei Y, Chen T, Banciu D, Cretoiu D, Xiao J. Circulating Exosomes in Cardiovascular Disease 2017; 998: 255-269.
Boulanger C, Loyer X, Rautou P, Amabile, N. Extracellular vesicles in coronary artery disease. Nature Reviews Cardiology 2017; 14(5).
Da Costa L, Arora P, Gacia-Bailo B, Karmali M, El-Sohemy A, Badawi A. The association between obesity, cardiometabolic disease biomarkers, and innate immunity-related inflammation in Canadian adults. Diabeter MEtab Syndr Obes 2012; 5: 347-355.
Deddebs H, Vrijsen K, Colijn J, Oerlemans M, Metz C, van der Vlist E, Nolte-′tHoen E, Ouden K, Jansen Of Lorkeers S, van der Spoel T, Koudstaal S, Arkesteijn G, Wauben M, van Laake L, Doevendans P, Chamuleau S, Sluijter J. Circulating Extracellular Vesicles Contain miRNAs and are Released as Early Biomarkers for Cardiac Injury. J. of Cardiovascular Translational Research 2016; 9: 291-301.
Fernandez-Valverde S, Taft R, Mattick J. MicroRNAs in B-Cell Biology, Insulin Resistance, Diabetes and Its Complications. Diabetes 2011: 60(7): 1825-1831.
Franca C, de Oliveira Izar M, do Amaral J, Tegani D, Fonseca F. Microparticles as Potential Biomarkers of Cardiovascular Disease 2015; 104(2): 169-174.
Gray M, Long C, Kalinyak J, Li H, Karliner J. Angiotensin II stimulates cardiac myocyte hypertrophy via paracrine of TGF-b1 and endothelin-1 from fibroblasts. Cardiovascular Research; 40(2): 352-363.
Jansen F, Nickenig G, Werner N. Extracellular Vesicles in Cardiovascular Disease. Circulation Research 2017; 120; 1649-1657.
Kalluri R, LeBleu V, The biology, function, and biomedical application of exosomes. Science 7 Feb. 2020 Vol. 367, Issue 6478.
Lin J, Li J, Huang B, Liu J, Chen X, Chen X M, Xu Y M, Huang L F, Wang X Z. Exosomes: novel biomarkers for clinical diagnosis. Scientific World Journal 2015; 657086.
Mateescu B, Kowal E J, van Balkom B W, et al. Obstacles and opportunities in the functional analysis of extracellular vesicle RNA—an ISEV position paper. J Extra-cell Vesicles 2017; 6: 1286095.
Pittenger, M, Discher D, Peault B, Phinney D, Hare J, Caplan A. Mesenchymal stem cell perspective: cell biology to clinical progress. NPJ Regen Med 2019; 4(22).
Pomatto M, Gai C, Deregibus M, Tetta C, Camussi G. Noncoding RNAs Carried by Extracellular Vesicles in Endocrine Diseases 2018; International Journal of Endocrinology: 2018.
Rahaman M, Mao J. Stem cell-based composite tissue constructs for regenerative medicine. Biotechnology and Bioengineering 2005; 91(3): 261-284.
Raposo G, Nijman H W, Stoorvogel W, et al. B lymphocytes secrete antigen-presenting vesicles. J Exp Med 1996; 183:1161-72.
Robbins P D, Dorronsoro A, Booker C N. Regulation of chronic inflammatory and immune processes by extracellular vesicles. J Clin Invest 2016; 126:1173-80.
Sharma P, Schiapparelli L, Cline H. Exosoms function in cell-cell communication during brain development 2013; Curr Opin Neurobiol; 23.
Sinha M, Ansell-Schultz A, Civitelli L, Hildesjo C, Larsson M, Lannfelt L, Ingelsson M, Hallbeck M. Alzheimer's disease pathology propagation by exosomes containing toxic amyloid-beta oligomers. Acta Neuropathol. 2018; 136(1): 41-56.
Thery C. Exosomes: secreted vesicles and intercellular communications 2011; F1000 Bio Rep; 3:15.
van der Pol E, Boing A N, Gool E L, Nieuwland R. Recent developments in the nomenclature, presence, isolation, detection and clinical impact of extracellular vesicles. J Thromb Haemost 2016; 14:48-56.
Vegter E, van der Meer P, de Windt L, Pinto Y, Voors A. MicroRNAs in heart failure: from biomarker to target for therapy 2016; 18(5).
Vulliet P R, Greeley M, Halloran S M, MacDonald K A, Kittleson M D. Intra-coronary arterial injection of mesenchymal stromal cells and microinfarction in dogs. Lancet. 2004; 363(9411):783-784.
Wang C, Zhang C, Liu L, Chen B, Li Y, and Du J. Macrophage-Derived mir-155-Containing Exosomes Suppress Fibroblast Proliferation and Promote Fibroblast Inflammation during Cardiac Injury. Molecular Therapy; 25(1): 192-204.
Wu P, Zhang B, Shi H, Qian H, Xu W. MSC-exosome: A novel cell-free therapy for cutaneous regeneration. Cytotherapy 2018; 20; 291-301.
Yao Y, Huang J, Geng Y, Qian H, Fan W, Liu X, Shang M, Nie S, Liu N, Du X, Dong J, Ma C. Paracrine Action of Mesenchymal Stem Cells Revealed by Single Cell Gene Profiling in Infarcted Murine Hearts. PLOS One 2015; 10: e0129164.
Zhang J, Li S, Li L, Li M, Guo, C, Yao J, Mi S. Exosome and Exosomal MicroRNA: Trafficking, Sorting, and Function. Genomics Proteomics Bioinformatics 2015; 13(1): 17-24.
Zitvogel L, Regnault A, Lozier A, Wolfers J, Flament C, Tenza D, Ricciardi-Castagnoli P, Raposo G, Amigorena S. Eradication of established murine tumors using a novel cell-free vaccine: dendritic cell-derived exosomes. Nat Med. 1998; 4: 594-600.

1. A method of isolating an exosome or an exosomal product from a tissue source comprising isolating the tissue source; and purifying the exosome or the exosomal product from the tissue source; wherein the exosome or the exosomal product comprises a micro RNA, a protein, and/or a cellular marker.

2. The method of claim 1, wherein the tissue source is selected from the group consisting of an umbilical cord tissue, a blood source, a perinatal source, a cell culture source, a MSC source, a pluripotent stem cell source, an induced pluripotent stein cell source, an mesenchymal stem cell source, a hematopoietic stem cell source, and a combination thereof.

3. The method of claim 2, wherein the cell culture source is either derived from a 2D cell culture source or a 3D cell culture source or a combination thereof.

4. The method of claim 3, wherein the 3D cell culture source comprises growing with a scaffold.

5. The method of claim 4, wherein growing with the scaffold further comprises adding nitric oxide and chitosan; wherein the cell culture increases in angiogenic and pro-migratory promoting activity; wherein the cell culture increases in vasoendothelial growth factor and miR126; and wherein the cell culture increases in ischemic muscle protection property.

6. The method of claim 1 further comprising adding Micelle to the tissue source; and filtrating the tissue source by centrifugation and/or size exclusion filtration.

7. The method of claim 6 wherein the Micelle used is poloxamer 188/407.

8. The method of claim 1, wherein purifying the exosome or the exosomal product comprises sterilizing the tissue source.

9. The method of claim 2, wherein isolating the tissue source comprises dissecting the umbilical cord tissue to expose a blood vessel from the umbilical cord tissue; removing the blood vessel from the umbilical cord tissue; rinsing the umbilical cord tissue with a saline solution to remove blood; placing the umbilical cord tissue in a solution; and exposing the umbilical cord tissue in the solution to UV light; and wherein purifying the exosome or the exosomal product from the tissue source comprises transferring the umbilical cord tissue to a tube or a container; and vortexing the umbilical cord tissue as a source of the exosome or the exosomal product.

10. The method of claim 1, wherein purifying the exosome or the exosomal product from the tissue source further comprising spinning the source at about 100×g for about 10 minutes to isolate a first supernatant; spinning the first supernatant at about 300×g for about 10 minutes to isolate a second supernatant; spinning the second supernatant at about 5,000×g for about 10 minutes to isolate a third supernatant; spinning the third supernatant at about 100,000×g for about 70 minutes to isolate a pellet; washing the pellet with a solution; and spinning the pellet at about 100,000×g for about 70 minutes to clean the pellet; and wherein the pellet comprises exosome or the exosomal product.

11. The method of claim 1, wherein the micro RNA is selected from the group consisting of miR21, miR 146a, miR 181c, miR 124a, miR 125b, miR 21, miR 23a, miR 125b, miR 145, miR 451, miR 133b, miR 126, miR 296, any variation of the aforementioned micro RNA, and a combination thereof.

12. The method of claim 1, wherein the micro RNA comprises miR21, miR 146a, miR 181c, miR 124a, miR 125b, miR 21, miR 23a, miR 125b, miR 145, miR 451, miR 133b, miR 126, and miR 296, and/or a derivative of any of the aforementioned micro RNA.

13. The method of claim 1, wherein the protein is selected from the group consisting of Annexin A1, B7-2, Clathrin, Flotillin-1, MFG-E8, MHC I, MEC II, Tsg 1, Tetraspanin, Neprilysin, a derivative of any of the aforementioned protein, and a combination thereof.

14. The method of claim 1, wherein the protein comprises Annexin A1, B7-2, Clathrin, Flotillin 1, MFG-E8, MHC I, MHC II, Tsg 1, Tetraspanin, and Neprilysin, and/or a derivative of any of the aforementioned protein.

15. The method of claim 1, wherein the cellular marker is selected from the group consisting of CD9, CD34, CD41, CD45, CD59, CD63, CD73, CD81, CD105, CD117, CD123, CD146, CD 166, a variation of any of the aforementioned cellular marker, and a combination thereof.

16. The method of claim 1, wherein the cellular marker comprises CD9, CD34, CD41, CD45, CD59, CD63, CD73, CD81, CD105, CD117, CD123, CD146, CD166, and a combination thereof.

17. The method of claim 1, wherein the exosome or the exosomal product has a diameter of about 20 nm to about 200 nm.

18. The method of claim 17, wherein the exosome or the exosomal product has a diameter of about 50 nm to about 100 nm.

19. The method of claim 1, Wherein the exosome or the exosomal product is among a plurality of exosomes or exosomal products; and wherein the plurality of exosomes or exosomal products comprise as a percentage of the plurality of about 69.0% to about 74.0% the cellular marker CD9, of about 28.1% to about 53.1% the cellular marker CD59, of about 1.2.3% to about 17.6% the cellular marker CD63, of about 62.7% to about: 83.0% the cellular marker CD73, or of about 62.2% to about 83.5% the cellular marker CD81.

20. The method of claim 19, wherein the exosome or the exosomal product is among a plurality of exosomes or exosomal products; and wherein the plurality of exosomes or exosomal products comprise as a percentage of the plurality of about 71.2% the cellular marker CD9, of about 40.0% the cellular marker CD59, of about 15.7% the cellular marker CD63, of about 73.2% the cellular marker CD73, or of about 73.3% the cellular marker CD81.

21-86. (canceled)
